(19) United States
(12) Patent Application Publication
(10) Pub. No.: US 2026/0033980 A1
(43) Pub. Date: Feb. 5, 2026

(54) Male Contraceptive Device with Secure Fit and One-Way Valve System

(71) Applicant: Davin S Cho, Los Angeles, CA (US)

(72) Inventor: Davin S Cho, Los Angeles, CA (US)

(21) Appl. No.: 18/795,152

(22) Filed: Aug. 5, 2024

(51) Int. Cl. A61F 6/04

(52) U.S. Cl. CPC A61F 6/04

(57) ABSTRACT

A male contraceptive device comprising a peanut-shaped elastic body having two opposing flanges, each with proximal and distal ends. An adhesive disposed on the inner surface ensures secure fit, while non-adhesive tabs at the distal ends facilitate application and removal. A centrally located double-layer reservoir collects fluids, with a one-way valve of three overlapping semicircular latex pieces that expand outwardly under pressure to allow fluid entry and fold inwardly to prevent leakage. An embodiment includes an anatomically-shaped divot for alignment and a pull tab for retrieval. Another embodiment features a three-flange, clover leaf configuration and a valve with outer, inner, and connective latex pieces. The device's unique combination of elements provides improved fit, comfort, ease of use, and contraceptive efficacy.

### BACKGROUND OF THE INVENTION

### Field of Invention

The present invention relates to the field of male contraceptive devices. More specifically, the invention pertains to a male contraceptive device featuring a peanut-shaped body with an adhesive inner surface, non-adhesive application tabs, a double-layer reservoir, and a one-way valve comprising overlapping latex pieces.

### Description of Related Art

Male contraceptive devices, such as condoms, have been widely used to prevent unwanted pregnancy and the spread of sexually transmitted infections. Conventional condoms typically feature a tubular elastic body designed to cover the penis during sexual intercourse, acting as a barrier to prevent the exchange of bodily fluids.

While conventional condoms are generally effective, they often suffer from issues related to fit, comfort, and ease of application. Poor fit can lead to slippage or breakage during use, compromising the contraceptive efficacy of the device. Additionally, some users find traditional condoms uncomfortable or cumbersome to apply, which may discourage consistent use.

Efforts have been made to address these shortcomings by incorporating various design features into male contraceptive devices. For example, some devices have employed adhesives to improve retention on the penis, as seen in U.S. Pat. No. 5,421,350. Others have explored the use of valves or reservoir structures to enhance functionality and comfort, as described in U.S. Pat. No. 7,654,265.

However, despite these advancements, there remains a need for a male contraceptive device that offers a combination of secure fit, user comfort, and ease of application while providing reliable contraceptive protection.

### BRIEF SUMMARY OF THE INVENTION

This summary is provided to introduce a selection of concepts, in a simplified format, that are further described in the detailed description of the invention. This summary is neither intended to identify key or essential inventive concepts of the invention nor is it intended for determining the scope of the invention.

The present invention is directed to a male contraceptive device that provides secure fit, user comfort, and ease of application while offering reliable contraceptive protection. The device features a peanut-shaped body formed of an elastic material, such as latex, and comprises two opposing flanges, each having a proximal end and a distal end. An adhesive is disposed on a majority of the inner surface of the peanut-shaped body to ensure a secure fit during use. Non-adhesive tabs are positioned at the distal ends of the flanges to facilitate easy application and removal of the device.

Centrally located within the peanut-shaped body is a double-layer reservoir designed to collect and retain fluids. Disposed within the reservoir is a one-way valve comprising three overlapping semicircular latex pieces. These pieces are configured to expand outwardly in response to pressure, allowing fluid to enter the reservoir, and fold inwardly after the pressure is removed to prevent fluid leakage.

In one embodiment, the device includes an anatomically-shaped divot at the distal end of the first flange to aid in proper alignment during application. A pull tab may also be attached to one of the flanges to facilitate retrieval of the device if dislodged.

Another embodiment of the invention features a peanut-shaped body with three flanges arranged in a clover leaf configuration, each having a non-adhesive tab at its distal end. The one-way valve in this embodiment comprises an outer latex piece, an inner latex piece concentrically attached to the outer piece, and three latex connective pieces attaching the inner piece to the outer piece at equidistant points. This valve configuration allows for opening and closing in response to pressure changes.

The combination of the peanut-shaped body, strategic adhesive placement, user-friendly tabs, double-layer reservoir, and innovative one-way valve system distinguishes the present invention from prior art devices. By addressing key issues related to fit, comfort, ease of use, and contraceptive efficacy, this invention represents a significant advancement in the field of male contraceptive devices.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1: A perspective view of an embodiment of a male contraceptive device with a peanut-shaped body and two flanges.

FIG. 2: The inner surface of an embodiment of the male contraceptive device, showing the adhesive area and valve position.

FIG. 3A: An aerial perspective view of an embodiment of the one-way valve with three overlapping semicircular latex pieces.

FIG. 3B: An embodiment of the one-way valve opening as the latex pieces are pushed up, allowing fluid to enter the reservoir.

FIG. 3C: An embodiment of the one-way valve closing as the latex pieces fold inwardly, preventing fluid from exiting the reservoir.

FIG. 4: Another embodiment of the male contraceptive device with an anatomical divot on one flange.

FIG. 5A: An embodiment of the valve comprising two overlapping latex pieces.

FIG. 5B: An embodiment of the valve opening as the two latex pieces unfold outwardly, allowing fluid to fill the reservoir.

FIG. 5C: An embodiment of the valve closing as the two latex pieces fold inwardly, preventing fluid leakage.

FIG. 6: A top plan view of an embodiment of a male contraceptive device with a three-leaf clover shaped body.

FIG. 7A: A top plan view of an embodiment of the one-way valve with an outer and inner latex piece connected by three latex connective pieces.

FIG. 7B: An embodiment of the valve opening as the inner latex piece lifts off the outer latex piece, allowing fluid to enter the reservoir.

FIG. 7C: An embodiment of the valve closing as the inner latex piece is pulled back against the outer latex piece, resealing the valve.

FIG. 8: A perspective view of an embodiment of the male contraceptive device with an hourglass-shaped body.

FIG. 9: A cross-sectional view of an embodiment of the male contraceptive device, showing the device with example dimensions.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

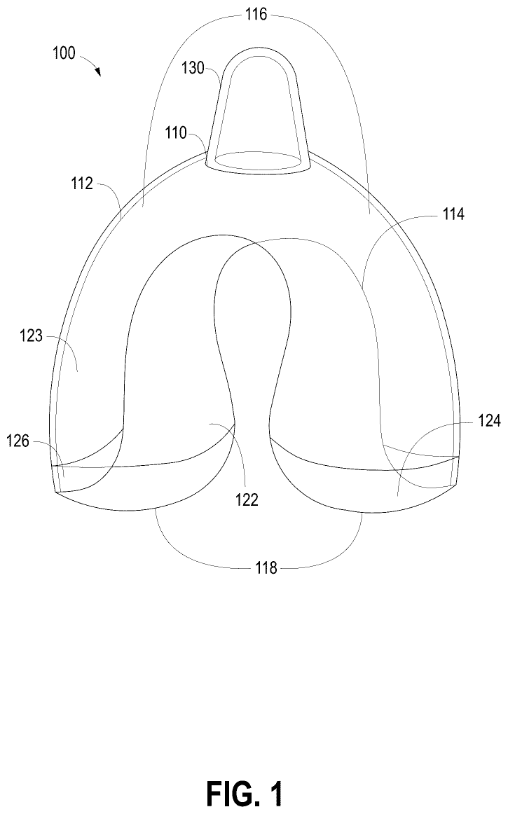

FIG. 1 illustrates a perspective view of a male contraceptive device 100. The device 100 comprises a peanut-shaped body 110 formed of an elastic material such as latex. As shown in FIG. 1, the peanut-shaped body 110 has a first flange 112 and a second flange 114, wherein each flange has a proximal end 116 and a distal end 118.

In one embodiment, an adhesive is disposed on a majority of an inner surface 122 of the peanut-shaped body 110. A first tab 126 without adhesive is disposed at the distal end 118 of the first flange 112, and a second tab 124 without adhesive is disposed at the distal end 118 of the second flange 114. According to an embodiment, the first and second tabs 124, 126 are configured to be wide enough to grab for easy application and removal of the device 100.

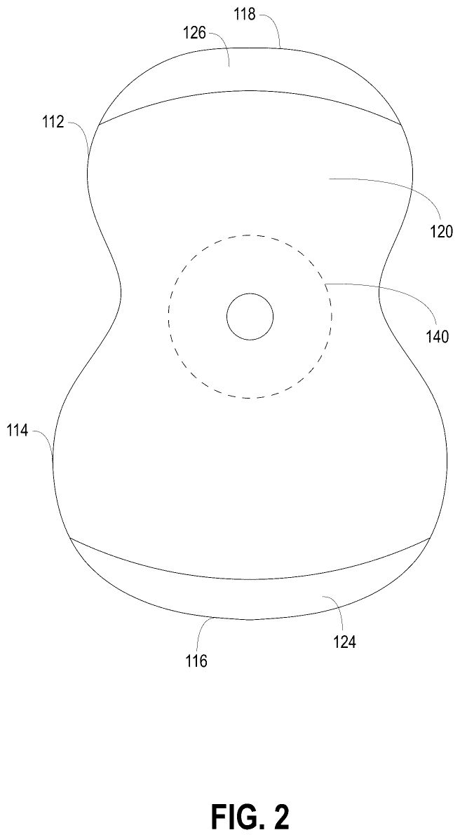

With reference to FIG. 2, the male contraceptive device 100 is shown from the inner surface 122. As depicted in FIG. 2, the ends of the peanut-shaped body 110, i.e. the first and second flanges 112, 114, have the tabs 124, 126 without adhesive on the top and bottom. The main back surface area of the peanut-shaped body 110 has the adhesive bordering the tabs 126, 124. A dashed line 132 in the center indicates the circumference of the double-layer reservoir 130 and shows where the adhesive stops. The center circle indicates the position of a one-way valve 140, which enables fluid to flow into the double-layer reservoir 130.

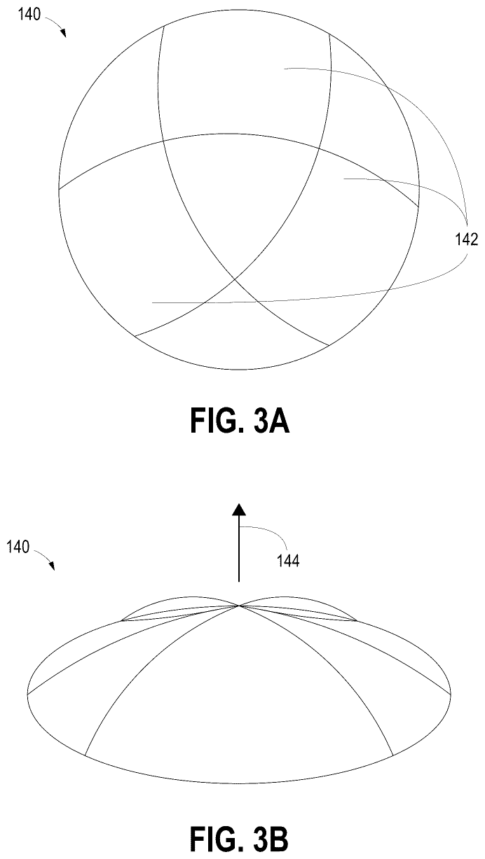

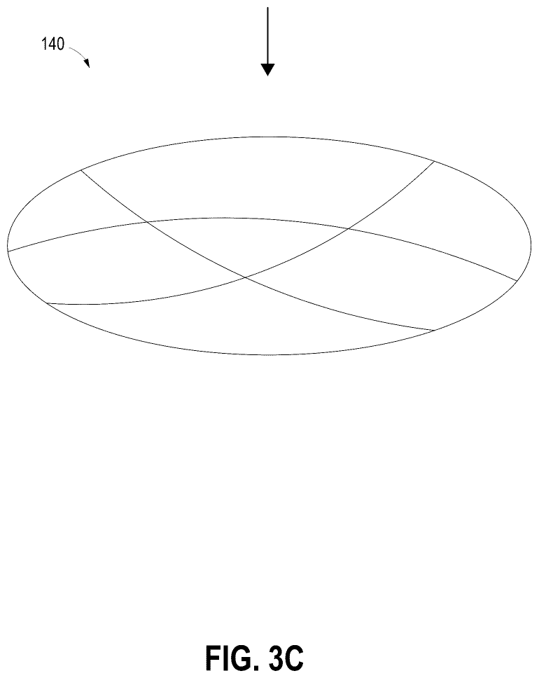

As illustrated in FIG. 3A, an aerial perspective view of the one-way valve 140 is shown, wherein the valve 140 is designed with the three overlapping semicircular latex pieces 142.

FIG. 3B depicts a perspective view of the valve 140 beginning to open as the three overlapping latex pieces 142 are pushed up, thereby creating gaps 144 and allowing fluid to enter the reservoir 130. This occurs in response to pressure applied to the valve 140.

In FIG. 3C, the latex pieces 142 of the valve 140 are shown sitting back down after fluid has moved into the reservoir 130. The latex pieces 142 fold inwardly, thereby preventing fluid from exiting the reservoir 130 after the pressure is removed.

In another embodiment, a pull tab (not shown) may be coupled to one of the first or second flanges 112, 114. The pull tab (not shown) is configured to facilitate retrieval of the device 100 in case it becomes dislodged.

In use, the tabs 126, 124 of the male contraceptive device 100 are grasped to apply the device, with the adhesive inner surface 122 adhering to the penis. During ejaculation, fluid enters the double-layer reservoir 130 by pushing open the valve 140. The fluid is then contained in the reservoir 130 as the valve 140 closes, thereby preventing backflow or leakage. After use, the tabs 124, 126 are again grasped to remove the device 100. If the device 100 becomes dislodged, the pull tab (not shown) may be used to retrieve it.

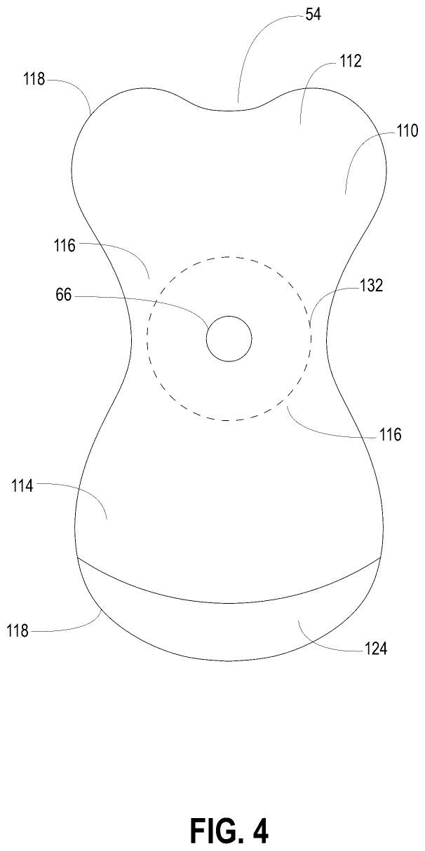

FIG. 4 illustrates another embodiment of the male contraceptive apparatus. The apparatus comprises a body 110 formed of a flexible material such as latex. As shown in FIG. 4, the body 110 has a peanut-shaped body with two opposing flanges 114, 112, each having a proximal end 116 and a distal end 118. An anatomical divot 54 is disposed at the distal end 118 of the flange 112 and is configured to align with a user's anatomy to facilitate proper application of the apparatus.

In one embodiment, an adhesive coating is disposed on a substantial portion of an inner surface of the peanut-shaped body of the body 110. A non-adhesive tab 124 is disposed at the distal end 118 of the flange 114 without the anatomical divot. The non-adhesive tab 126 is configured to be wide enough to facilitate easy application and removal of the apparatus.

With reference to FIG. 4, a double-layer reservoir 130 is disposed centrally on the opposing outer surface 123 of the body 110. The double-layer reservoir 130 is disposed in a circular shape. A valve 66 is disposed on the reservoir 130. The adhesive coating extends to a boundary indicated by a dashed line 132 circumscribing the reservoir 130, wherein the dashed line 132 further indicates where the adhesive coating stops and the reservoir 130 begins.

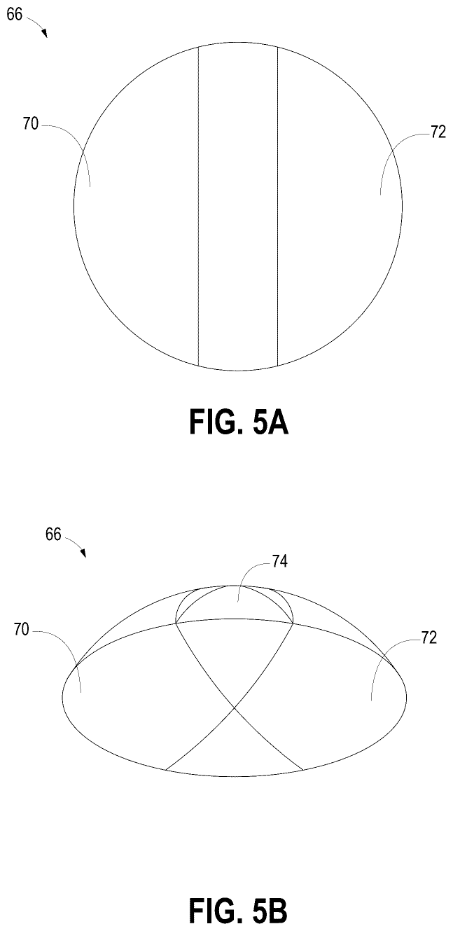

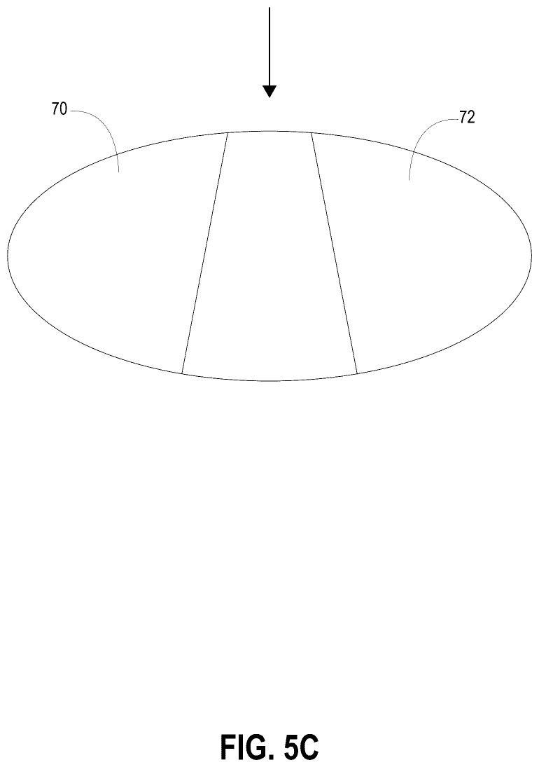

As depicted in FIG. 5A, the valve 66 comprises two overlapping latex pieces 70, 72.

FIG. 5B illustrates a perspective view of the valve 66 taken along line 5B-5B of FIG. 5A. The two overlapping latex pieces 70, 72 are configured to unfold outwardly, thereby creating an opening 77 to allow fluid to fill the reservoir 130, as shown by the gap 74 created between the latex pieces 70, 72.

FIG. 5C depicts a perspective view similar to FIG. 5B, showing the two overlapping latex pieces 70, 72 folded inwardly to reseal and prevent fluid leakage after fluid has moved into the reservoir 130.

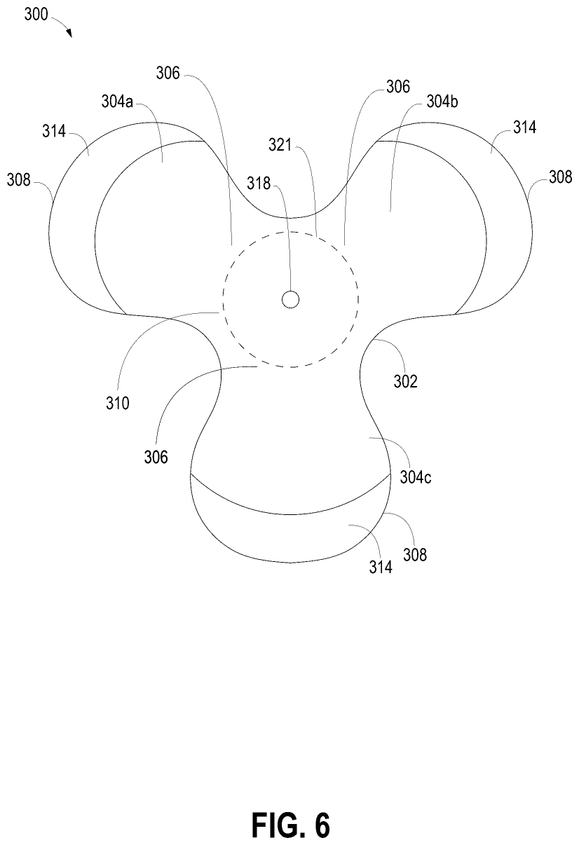

FIG. 6 illustrates a top plan view of a male contraceptive device 100 according to another embodiment of the present invention. The device 100 comprises a body 302 formed of an elastic material such as latex or polyurethane. As shown in FIG. 6, the body 302 has a three-leaf clover shape with three flanges 304a, 304b, 304c arranged symmetrically around a central point, wherein each flange 304a, 304b, 304c has a proximal end 306 connected to the central point and a distal end 308.

In one embodiment, an adhesive is disposed on a majority of an inner surface of the body 302, covering a substantial portion of each flange 304a, 304b, 304c. The adhesive 310 is configured to adhere the device 100 to the glans of the penis during use. As depicted in FIG. 6, at the distal end 308 of each flange 304a, 304b, 304c, there is a tab 314 that is free of adhesive, similar to the tabs 126, 124 in FIG. 2. The non-adhesive tabs 314 are configured to facilitate application and removal of the device 300 by providing a gripping surface.

In the embodiment shown in FIG. 6, a double-layer reservoir 130 is disposed at the center of the body 302 on the outer surface 123, at the point where the three flanges 304a, 304b, 304c connect. The reservoir 316 is configured to collect and retain fluid during use. The dashed line 321 further indicates where the adhesive coating stops and the double layer reservoir 130 begins.

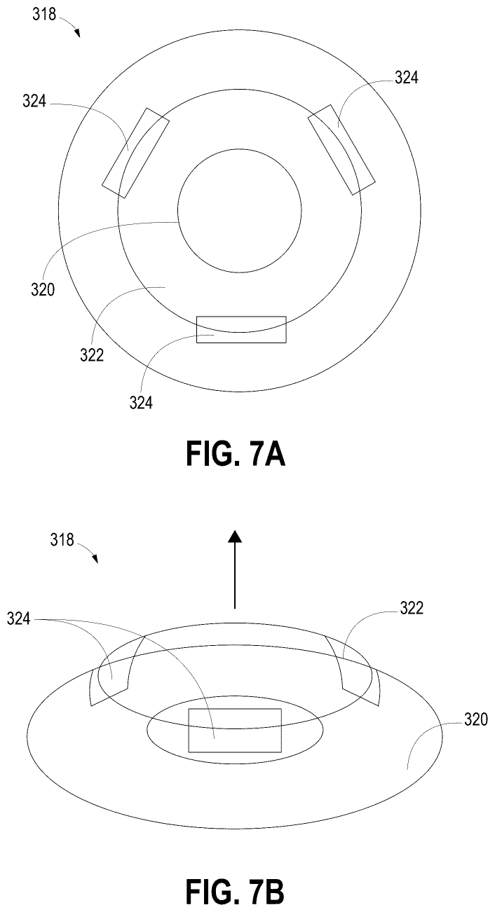

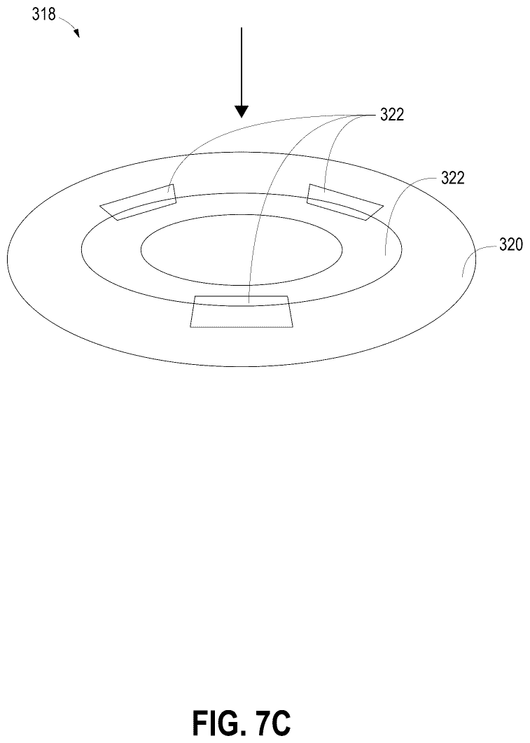

As illustrated in FIG. 6, disposed at the center of the male contraceptive device is a one-way valve 318. FIG. 7A shows a top plan view of the valve 318, while FIGS. 7B and 7C depict perspective views of the valve 318 in open and closed configurations, respectively.

In one embodiment, the valve 318 comprises an outer latex piece 320 and an inner latex piece 322 concentrically attached to the outer latex piece 320. Three latex connective pieces 324 attach the inner latex piece 322 to the outer latex piece 320 at points equidistantly spaced around the circumference of the inner latex piece 322, approximately 120 degrees apart with respect to each other.

As shown in FIG. 7B, the valve 318 is configured to open by the inner latex piece 322 lifting off the outer latex piece 320 in response to fluid pressure, thereby allowing fluid to enter the reservoir 130. The connective pieces 324 stretch to permit this lifting motion.

As depicted in FIG. 7C, the valve 318 is configured to close by the inner latex piece 322 being pulled back against the outer latex piece 320 by the elastic recoil of the connective pieces 324 once fluid pressure subsides, thereby resealing the valve 318 to prevent fluid from exiting the reservoir 316.

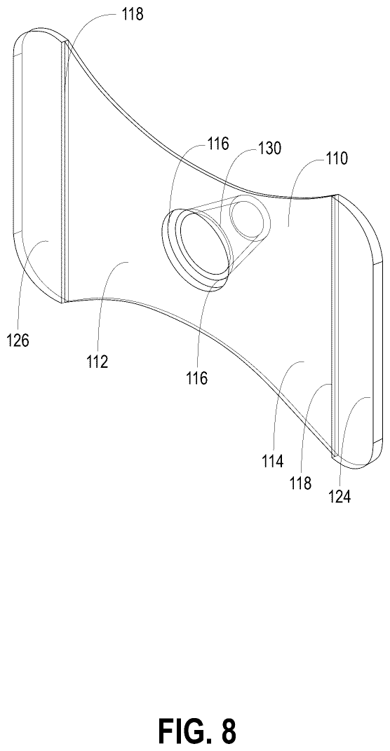

FIG. 8 illustrates a perspective view of an embodiment of the male contraceptive device 100. The device 100 comprises a body 110 formed of an elastic material such as latex. In this embodiment, the body 110 has an hourglass-shaped portion with two opposing flanges 112, 114, each having a proximal end 118 and a distal end 118, wherein the flanges 112, 114 taper outwardly from the proximal ends 116 to the distal ends 116.

As shown in FIG. 8, the double layer reservoir 130 protrudes from an outer surface 123 of the body 110, opposite the inner surface 122 with the adhesive coating. The double layer reservoir 130 is configured in a dome-shaped configuration. A valve 226, which may be any of the three types described in FIGS. 3A-3C, 5A-5C, or 7A-7C, is disposed on the reservoir 130.

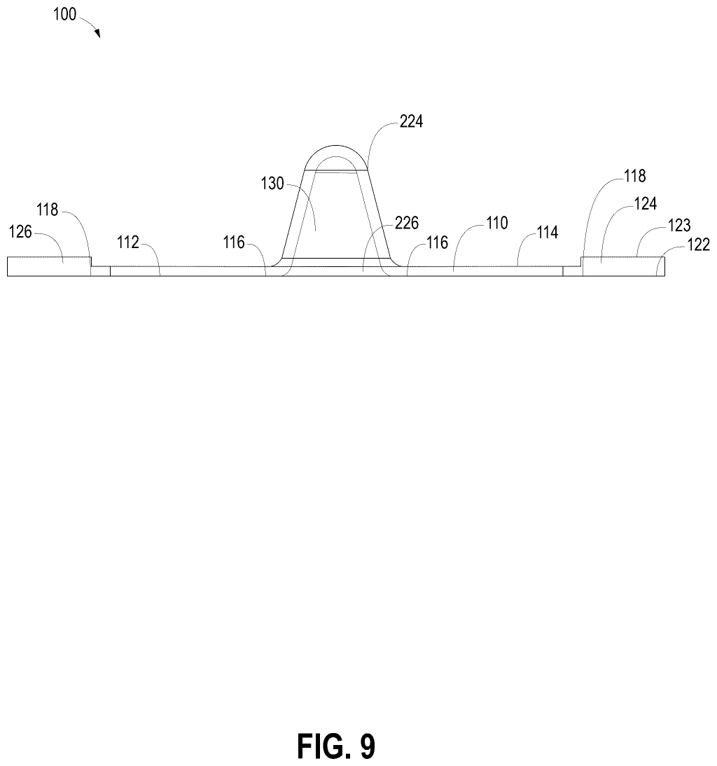

With reference to FIG. 9, a cross-sectional view of the male contraceptive device 100 taken along a lateral plane is depicted, showing the device 100 with example dimensions. The double layer reservoir 130 protrudes from the outer surface 123 of the device 100, wherein the walls 228 of the reservoir 130 form a dome-shaped cone. The walls 228 extend at an angle of approximately 23.73 degrees from a plane perpendicular to the back side of the device 100.

As illustrated in FIG. 9, the non-adhesive tabs 126, 124 disposed at the distal ends 118 of the flanges 112, 114 have a width of approximately 10 mm, spanning the ends of the flanges 112, 114. The tabs 126, 124 have a thickness of approximately 2 mm, while the body 110 portion of the flanges 112, 114 has a thickness of approximately 1 mm. It should be noted that these dimensions are provided as an example embodiment and are not intended to limit the scope of the invention, as they may vary for different sizes of the device 100 to accommodate different male anatomies.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A male contraceptive device, comprising:
a. a peanut-shaped body formed of an elastic material and having a first flange and a second flange, each flange having a proximal end and a distal end;
b. an adhesive disposed on a majority of an inner surface of the peanut-shaped body;
c. a first tab without adhesive disposed at the distal end of the first flange and a second tab without adhesive disposed at the distal end of the second flange, the first and second tabs configured for application and removal of the device; and
d. a reservoir disposed at a center of the peanut-shaped body.

2. The male contraceptive device of claim 1, further comprising a one-way valve disposed on the reservoir, the valve comprising three overlapping semicircular latex pieces configured to expand outwardly to permit fluid to enter the reservoir and fold inwardly to prevent fluid from exiting the reservoir.

3. The male contraceptive device of claim 1, wherein the tabs are configured to be wide enough to grab for easy application and removal of the device.

4. The male contraceptive device of claim 1, wherein the reservoir is a double-layer reservoir.

5. The male contraceptive device of claim 1, wherein the three overlapping semicircular latex pieces of the one-way valve are configured to expand outwardly to permit fluid to enter the reservoir in response to pressure applied to the valve.

6. The male contraceptive device of claim 5, wherein the three overlapping semicircular latex pieces are further configured to fold inwardly after the pressure is removed to prevent fluid from exiting the reservoir.

7. The male contraceptive device of claim 1, wherein the first flange comprises an anatomically-shaped divot disposed at the distal end to facilitate alignment of the device during application.

8. The male contraceptive device of claim 1, further comprising a pull tab attached to one of the first or second flanges, the pull tab configured to facilitate retrieval of the device if dislodged.

9. The male contraceptive device of claim 1, wherein the peanut-shaped body comprises three flanges arranged in a clover leaf configuration, each flange having a tab without adhesive disposed at a distal end thereof.

10. The male contraceptive device of claim 9, wherein the one-way valve comprises an outer latex piece, an inner latex piece concentrically attached to the outer latex piece, and three latex connective pieces attaching the inner latex piece to the outer latex piece at points equidistantly spaced around the circumference of the inner latex piece, the valve configured to open by the inner latex piece lifting off the outer latex piece in response to pressure and to close by the inner latex piece being pulled back against the outer latex piece by the three latex connective pieces.

11. The male contraceptive device of claim 1, wherein the body is configured in a three-leaf clover shape, with each leaf having a proximal end and a distal end.

12. A contraceptive apparatus, comprising:
a. a body formed of a flexible material and having a peanut-shaped body and an anatomical divot, the peanut-shaped body comprising two opposing flanges each having a proximal end and a distal end;
b. an adhesive coating disposed on a substantial portion of an inner surface of the peanut-shaped body of the body;
c. a non-adhesive tab disposed at the distal end of each of the two opposing flanges, the non-adhesive tabs configured to facilitate application and removal of the apparatus;
d. a reservoir disposed centrally within the peanut-shaped body of the body; and
e. a valve disposed on the reservoir, the valve comprising two overlapping latex pieces configured to unfold outwardly to allow fluid to fill the reservoir and fold inwardly to reseal and prevent fluid leakage.

13. The contraceptive apparatus of claim 12, wherein the flexible material is latex.

14. The contraceptive apparatus of claim 12, wherein the non-adhesive tabs are configured to be wide enough to facilitate easy application and removal of the apparatus.

15. The contraceptive apparatus of claim 12, wherein the reservoir is disposed in a circular shape centrally within the peanut-shaped body of the body.

16. The contraceptive apparatus of claim 12, wherein the valve comprises three overlapping latex pieces configured to unfold outwardly to allow fluid to fill the reservoir and fold inwardly to reseal and prevent fluid leakage.

17. The contraceptive apparatus of claim 12, further comprising a pull tab disposed at the distal end of one of the two opposing flanges, the pull tab configured to facilitate retrieval of the apparatus if dislodged from a user.

18. The contraceptive apparatus of claim 12, wherein the anatomical divot is disposed at the proximal end of the peanut-shaped body and is configured to align with a user's anatomy to facilitate proper application of the apparatus.

19. The contraceptive apparatus of claim 12, wherein the adhesive coating extends to a boundary indicated by a dashed line circumscribing the reservoir, the dashed line further indicating where the adhesive coating stops and the reservoir begins.

20. The contraceptive apparatus of claim 16, wherein the three overlapping latex pieces are spaced approximately 120 degrees apart with respect to each other around the circumference of the valve.
